(19) United States
(12) Patent Application Publication
(10) Pub. No.: US 2017/0000997 A1
(43) Pub. Date: Jan. 5, 2017

(54) Tissue Needle With Side Aperture for Access To Target Location

(71) Applicants: NISHANT JALANDHARA, Reno, NV (US); PRIYANKA JALANDHARA, Reno, NV (US)

(72) Inventors: NISHANT JALANDHARA, Reno, NV (US); PRIYANKA JALANDHARA, Reno, NV (US)

(21) Appl. No.: 14/791,466

(22) Filed: Jul. 5, 2015

(51) Int. Cl. A61M 39/02

(52) U.S. Cl. CPC A61M 39/0247; A61M 2039/0205; A61M 2039/0255; A61M 2039/0261; A61M 2039/0291; A61B 2017/3456

(57) ABSTRACT

A needle suitable for use by inserting thru tissue for gaining access to target location. A needle is provided which has improved functionality and reducing complexity of procedure. The needle is comprised of an end with side aperture towards the tip portion, which is connected via conduit portion to the needle port. In one embodiment, the needle is comprised of a hollow needle with a port and a distal atraumatic end towards which there is a single aperture on the side wall, wherein a wire can be moved within the needle without damaging the wire. In other embodiment, the needle is comprised of a hollow needle with more than one port connected to tip portion towards which there are multiple apertures on the side wall, wherein a contrast or fluid injected from one port and wire can be inserted and removed from another port of the needle.

### BACKGROUND OF THE INVENTION

The present invention is in the technical field of Medicine. More particularly, the present invention is in the technical field of medicine where there is need to gain access to target location, body cavity or vessel lumen by inserting a needle. Currently one of the more common ways to gain access to target location is to insert needle in the body. Those needles could be sharp or blunt ended based on the target location and purpose of use. It is a very common practice to use a needle system with cannula and stylet (Wiley, 2012) or one with a side aperture. (Richard Kulkashi, 1992) Upon insertion to desired area, the stylet is removed and location confirmed by either the flow of desired fluid (e.g. spinal fluid in spinal cord placement, pleural fluid in pleural placement, blood in vascular placement) or injecting contrast or similar material (e.g. contrast injection in peritoneal cavity to confirm peritoneal placement) into the needle port.

This process of placing the needle at target site is followed by the objective of carrying out the procedure e.g. collect samples (spinal fluid, pleural fluid) or inject drugs (peritoneal space) or pass a wire to secure the access to location (in blood vessels or other body cavities). Most of the current needles in market have openings at the end (e.g. Hawkins needle) which, after removal of the stylet could injure the underlying organs (e.g. due to unexpected movements by operator as connecting/disconnecting syringe or movements by patient subject as with breathing or coughing). There are also needles currently available in market with side opening but they do not allow passage and withdrawal of wire through the side openings as they have sharp edges on side openings or are angled which is not ideal for movement of wire in and out of needle. (Walter A. Z0hmann, 2003; Peter John Crocker, 2005; Johnson, 1987)

The above-described needles are far from ideal. Generally speaking the following steps involved using current needle design with cannula during needle placement to target location: a) insertion of needle into tissue, b) removal of stylet, c) attachment of syringe to needle port, d) injection of contrast/aspiration of fluid thru the port to confirm location, e) detachment of syringe from port, f) insertion of desired wire thru port to gain and secure access to target location, and g) removal of needle over the wire, leaving one end of the wire securely at target location. Performing these multiple steps has its own limitations, namely: a) longer the procedure time as multiple steps are need to be performed (removing stylet, hooking/unhooking syringes), b) Increase risk of procedure complications (organ/vessel perforation/injury) due to risk of needle movement while performing any of the above steps or movement by patient, c) complications arising while removing wire from the needle due to wire getting stuck in needle or breaking inside the patient's body due to wire getting caught because of direction of needle aperture, d) increased complexity of the procedure as multiple discrete steps needs to be followed sequentially, and, e) increased chances of error due to increased complexity (e.g. injection of medicine instead of contrast to confirm location, insertion of wire without confirming location while needle is not in right location etc.) Using a Veress needle in an above described method to gain access to peritoneal sac, there is a 15% first pass failure rate, 0.3% morbidity rate, and 0.07% mortality rate, principally from either failing to fully penetrate into the peritoneum or puncturing vital organs and vessels. (U.S. Pat. No. 8,608,697 B2, 2013)

Previously needles have been described which use stylet, where the stylet has to be removed after insertion. (Young, 1982; Anderson, 1991; Teves, 1988). These needle designs would cause problems as once the stylet is removed, the pointed hollow needle tip would rest on the tissue. During the process of attaching/detaching syringe or movement by operator or patient, the pointed hollow needle tip could lead to complications, including damage to the tissue or organs around the needle tip.

Another type of needle (Johnson, 1987) has also been described with side aperture and blunt tip. This needle would provide the needed blunt end but would not facilitate passage of wire or similar structure to and from the needle lumen due to the angle or the aperture or wire being caught in the aperture. Even when those holes are created towards the end of the needle, their deflection angle is not conducive enough to permit smooth passage of wire. Given that those holes are punched onto the lateral surface of the needle, it would potentially lead to the wire getting catch on it and/or break it, further leading to another complication for its use.

This invention seeks to provide an improved type of tissue needle by allowing the two primary functions of the needle (i.e. tissue penetration and fluid transfer) while also allowing passage of wire and hence simplifying the entire process of needle insertion and gaining access to target location without the disadvantages and compromises forced by existing needle designs.

### SUMMARY

The present invention addresses the above and other needs by providing a needle having no stylet, a side aperture at the insertion end, and which permits transfer of fluid, gas and substances like wire thru it, all by simplifying and reducing the multiple steps of the overall procedure as described above.

In one embodiment of the needle, the needle body has a blunt end with side aperture partially covering the tip, designed and directed in a way that it would permit the passage and withdrawal of wire from the needle. The location of the aperture to partially cover the tip would also be conducive to remove needle over the wire without the distal end of needle getting stuck in tissue upon withdrawal of needle. The needle could also have a solid body at the needle tip portion after last aperture so that it facilitates movement of wire in and out of the aperture and not letting the wire getting caught within the needle conduit portion whereby the margins of the aperture are smoothened.

In one embodiment, the needle has two ports at the top. One to attach a syringe and other to possibly run a wire once the position of the needle tip portion is confirmed at target location without the need to detach the syringe at the first port. It is thus a feature of the present invention to provide a needle which has the composite properties of reducing the time and complexity of doing the procedure and provide better stability and functionality to the needle system and potentially simplifying the process and reducing the complications.

It is yet another feature of the present invention to provide a needle which can be used in combination with a wire which may be inserted and rapidly deployed to secure access to the target location such that the side aperture has two sections connected to two different ports respectively and each connected to a syringe and a wire respectively so that upon insertion, the syringe can be used to confirm the locations of needle tip followed by deployment of the wire.

In many embodiments, the distal tip of the needle comprises a tapered profile, and the distal tip of the needle may comprise an atraumatic needle tip.

In some embodiments, the needle may comprise indicia over the proximal portion thereof. The indicia may indicate the position of the distal end of the needle relative to the marks. The indicia may comprise a scale form printed or etched on a surface of the needle. In specific embodiments, the indicia may comprise a color change on the needle.

In one embodiment of the needle, the needle body has a blunt end with side aperture just proximal to the tip, designed and directed in a way that it would permit the passage and withdrawal of wire from the needle. The location of the aperture to be just proximal to the tip would be conducive to create just enough space by the needle tip being pushing the tissue, so as to allow for the passage of the wire. The needle could also have a solid body at the needle tip portion after last aperture so that it facilitates movement of wire in and out of the aperture and not letting the wire getting caught within the needle conduit or aperture portion, whereby the margins of the aperture are smoothened.

In some embodiments, the needle may have a stylet placed from one port while a syringe attached to another port, while both are connected to the common conduit area whereby as soon as needle is placed, the stylet removed from first port and location confirmed by injection of contrast in second port followed by insertion of wire in the first port to secure location at target tissue location.

In specific embodiments, at least the needle or various components of the needle may comprise of tungsten, rhenium, tantalum, palladium, tungsten-carbide, stainless steel, molybdenum-rhenium, molybdenum or cobalt-chromium.

Thus, the present invention provides an improved needle which can be used advantageously in multiple different procedures where access to tissue, vessel lumen or cavity is needed.

### BRIEF DESCRIPTION OF THE DRAWINGS

The above and other aspects, features and advantages of the present invention will be more apparent from the following more particular description thereof, presented in conjunction with the following drawings, wherein:

FIG. 1 shows, in accordance with the present invention, the system comprising the needle in a front elevation view.

FIG. 2 shows a right side elevation view of the needle illustrated in FIG. 1;

FIG. 3 is a left side elevation view of needle illustrated in FIG. 1;

FIG. 4 is a section taken generally along line C-C thereof in FIG. 1;

FIG. 5a is a section taken generally along line A-A in FIG. 1;

FIG. 5b is a section taken generally along line A-A in FIG. 1 also representing the movement of wire within the needle

FIG. 5c is a section taken generally along line A-A in FIG. 1 depicting the angle of the direction of the aperture opening.

FIG. 6 is a tip view (view from the needle tip where the longitudinal axis of the needle body projects into and behind the page) of the needle illustrated in FIG. 1

FIG. 7 is the detailed view ‘B’ of the needle tip illustrated in FIG. 1

FIG. 8 shows another embodiment of invention of FIG. 1

While invention will be described in connection with a certain preferred embodiment, it will be understood that it is not intended to limit this invention to that particular embodiment. On the contrary, it is intended to cover all alternatives, modifications, and equivalents as may be included within the spirit and scope of invention as defined by the appended claims.

### DETAILED DESCRIPTION OF THE EMBODIMENTS

The following description is of the best mode presently contemplated for carrying out invention's work. This description is not to be taken in a limiting sense, but is made merely for the purpose of describing the general principles of the current embodiment. The present invention provides, in one aspect, an improved needle that may be used in various applications such as abdominal cavity insertion or lumbar puncture. It will be appreciated by those skilled in the art that the needle of the present invention is not limited to aforementioned applications but may be used for other applications that utilizes needle, such as, for example, pleural fluid tapping.

One version of the invention is better understood, with reference to the figures. Turning now to the drawings, FIGS. 1, 2 and 3 show various external views of the needle, generally designated 10, in accordance with the present invention FIG. 1 shows a perspective front view of an embodiment of a three-dimensional form of a needle comprising of a tip portion (20) incorporating at least a tip (21), at least one aperture (22), conduit portion (30) incorporating at least one barrel section (32) in fluid communication (31) with the aperture (22) on one end and one or more port (40) on the other end. The lateral aperture (22) is designed and placed towards the tip (21) so as to help with the passage of wire as described below. The needle being relatively solid and strong while it could provide a rounded atraumatic point (21) to prevent organ damage.

The needle is suitable for use in many types of applications involving the transfer of fluids, gases or a combination thereof or inserting wire to secure access to target location. Such substances may be liquids or gases, including solutions, colloids, and suspensions of particulate matter in fluids or gases or materials like wire, polymers or a combination of above. The advantageous properties of the needle in construction and composition that make it strong but relatively atraumatic in use, causing less damage to the tissue it penetrates, make it suitable for transferring fluids to and from bodies, or run wires to and from bodies, as well as many other applications that will become evident to a broad range of users.

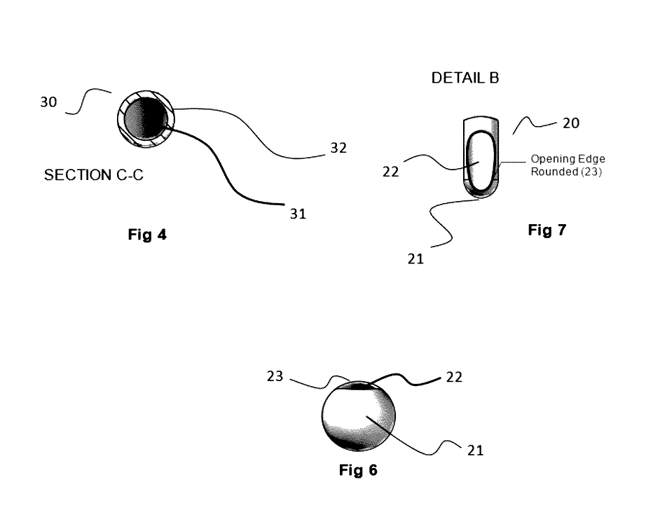

Gaseous, fluidic or solid materials are transferred from port (40) through the conduit portion (31) which in turn is connected to the aperture (22) and vice versa. It will be understood that one of more port (40) may act as an inlet or outlet for material, which may move in either direction through the conduit portion (31). Referring now to FIG. 4, it shows a cross section view at point C-C of the needle 10 as shown in FIG. 1. The cross section comprises of the conduit portion (31) and barrel section (32). It will be understood that the conduit may take other embodiments, such as being comprised of more than one passage. Multiple passages may be concentrically disposed of side-by-side.

In use, the needle penetrates the skin and tissue which seals on the barrel (32) above the aperture, as it passes through it. When the needle has penetrated to the target location the fluid is injected into the conduit portion (31) via port or substance seen flowing out of the port (40) to confirm location. The fluid or air could also be sucked out of the target location by applying negative pressure at the port (40).

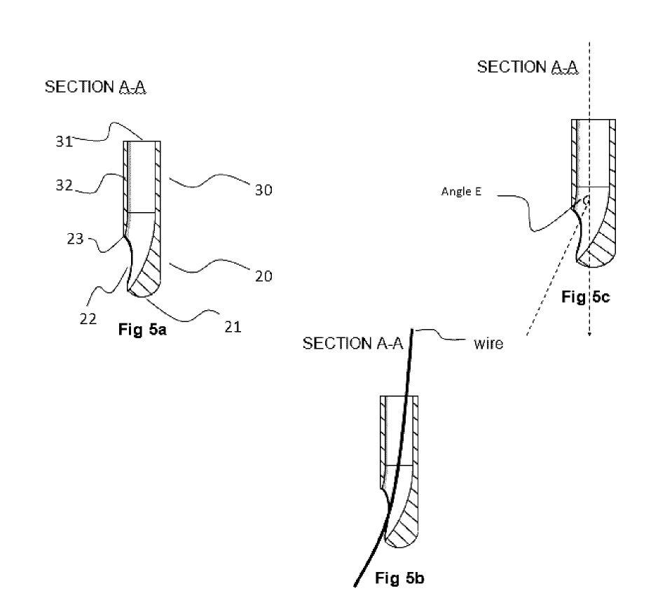

Referring now to FIGS. 5a, 6 and 7, these show a side view, point view and a front view respectively, of an embodiment of needle tip portion (20) which can be made of metal or a suitable plastics or polymer or an alloy or a combination of such materials, and comprises a needle tip (21) and an aperture (22) close to it. The needle tip (21) can be blunt or sharp. In use, the needle tip (21) penetrates the skin and there is preferably a minimum tissue cutting and displacement by the needle (10) itself. In use the small hole formed in skin and tissue by the tip (21) is smoothly extended along the path and elongate section with minimum of sharp edges or sharp changes in direction to catch or snag tissue. The needle tip portion (20) can be made of one or more different materials where the separate parts are joined together. The rounded end tip (21) is a surprisingly efficient means of atraumatic insertion and opening up tissue as the needle tip (21) penetrates the tissue. Preferably the needle comprises of a blunt point at a distal end as illustrated herein. However, the scope of the Invention includes that the distal end includes alternative shapes (e.g. sharp or pointed based on desired use) depending on the intended use.

Referring now to FIGS. 5A, 5B and 5C, there shows a plan side view of an embodiment of needle tip portion (20) which comprises a needle tip (21) and an aperture (22) formed in it. The aperture is placed and angled in such a way that when a wire or similar structure is passed into the conduit portion (31) it would facilitate the passage of wire through the aperture (22). The same aperture shape and angle would also help facilitate the removal of the wire from the aperture (22) and conduit (31) without getting the wire caught or kinked by the aperture edges (23) or direction of aperture opening (Angle E). Generally the conduit portion (31) leading up to the aperture (22) is angled (Angle E) at more than 110 degree angle to the axis of the needle to facilitate the movement of wire or similar structure without getting it kinked or caught on the aperture edges (23). The position of the aperture (22) closer to the needle tip (21) would also facilitate the removal of the needle over the wire without the distal end of the needle after the last aperture getting stuck in tissue upon withdrawal of needle. It will be understood that the aperture (22) may take other embodiments, such as being more than one aperture on the needle end and multiple apertures may be disposed concentrically or side-by-side.

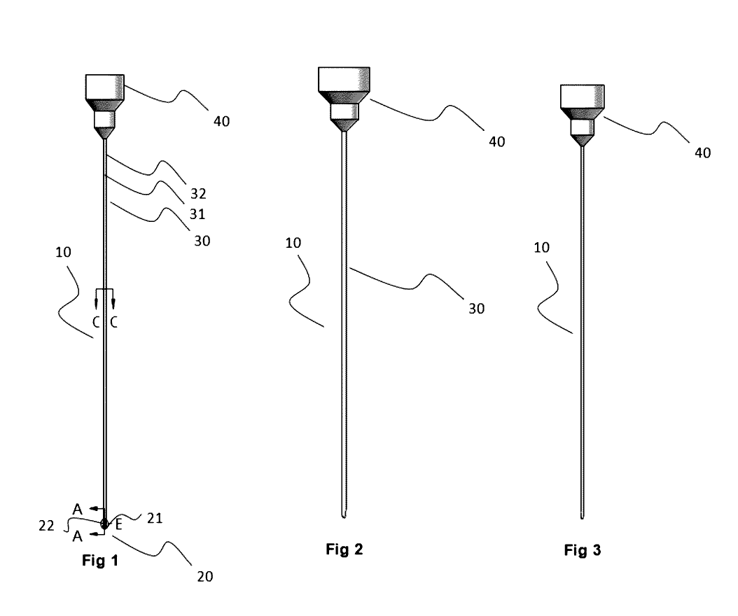

Referring now to FIG. 6, shows a view from the end point (20) of the needle tip (21) perpendicular to the side view in FIG. 2. From the viewer's perspective the longitudinal axis of the needle tip projects into and behind the page. This figure captures the idea that the aperture (22) is placed closer to the needle tip (21) so that it partially includes the tip so that upon tissue penetration still rendering the needle atraumatic while facilitating the exit of wire very close to the tip portion which is in line with the current longitudinal axis of the needle.

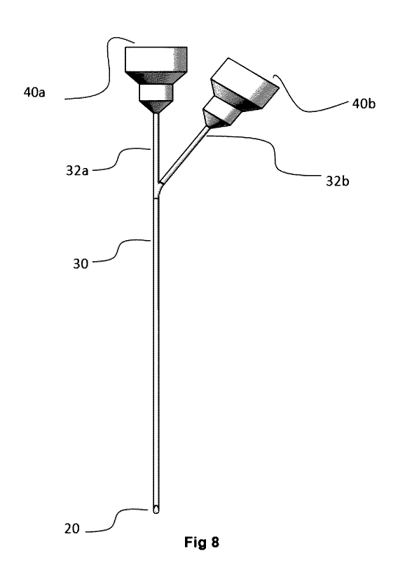

Referring now to FIG. 8, shows a plan view of a further embodiment of a three-dimensional form of the invention and in which there is a needle tip portion (20) incorporating at least a needle end (21), at least one aperture (22) for substance transfer, conduit portion (30) incorporating at least one barrel section (32) in fluid communication (31) with aperture (22) on one end and diverging into two barrel sections (32a and 32b) leading to their respective ports (40a and 40b) on the other end. The syringe can be mounted through one port (40a) and a wire or similar material passed thru another port (40b). Such an embodiment would permit passage of wire thru port (40b) to the conduit portion (31) to the aperture (22) as soon as the aperture (22) location is confirmed at target location by injection of contrast material or fluid or flow of fluid through port (40a) without the need to disconnect the syringe from the first port (40a).

Preferably the needle is comprised of a stainless steel or polymeric material. The needle may need reinforcement component that can be made of materials such as stainless steel, tungsten, elgiloy, platinum molybdenum, iridium or nitinol and/or other metals. It could also comprise alloys, e.g. platinum-iridium, molybdenum-rhenium, tungsten-rhenium, platinum-tungsten and/or other alloys. The materials could also be polymers such as Kevlar, Dacron, PEEK and/or other polymers. The needle can also comprise of at least one separate component made of one or more different materials and which the separate parts are joined together and made of different materials as mentioned above. Preferably the material is suitable for injection molding or compression molding of any type, for example fluid-assisted molding, two-shot molding, thermoplastic or thermosetting molding. The needle can be made by process steps comprising closing the tip of needle (20), forming a primary angle onto tip porting (20), processing by mechanical or chemical means to shape and smoothen the conduit portion (31) leading up to the aperture (22) and the edges of the tip and all apertures. Preferably the process includes the steps of providing a surface to reduce friction and/or retain lubricant at the surface. It can include using laser welding the different parts of the needle.

In some embodiments, the needle could comprise of polymer, with or without, a metal stylet to provide inner strength, while being less brittle and being less traumatic to the penetrated tissue. Such polymer needle would also require more cycles and/or force to break than a stainless steel needle. Also, known injection molding or folding techniques can make the polymer needle easier to manufacture.

The scope of the invention is not limited to having a single port and aperture in a needle tip as shown in FIGS. 1 thru 3. Other embodiments are within the scope, including one shown in FIG. 8. The scope of the invention includes that even further embodiments that are not illustrated in this document, including a plurality of apertures and a plurality of ports with various combinations of connections.

For many uses the needle has a length of 2 to 200 mm. The needle can be used with any injection device such as a syringe, pen, etc. and the length of the needle is chosen for the application of the needle. Examples of use are to add or remove substance during procedures including paracentesis, pleurocentesis, and lumbar puncture, gaining access to any body cavities like abdomen, pleural, spinal or into any blood vessels. It would also include but not limited to sub-cutaneous, intra-muscular, intra-venous, into bone, into joint, into eye, into any organ, and for any tissue penetration. The needle can also be used to add or remove substance for medicinal or diagnostic or other purposes for human or animal or other applications.

While the invention herein disclosed has been described by means of specific embodiments and applications thereof, numerous modifications and variations could be made thereto by those skilled in the art without departing from the scope of the invention set forth in the claims.

In broad embodiment, the present invention is that of a needle (10) suitable for use in many types of applications comprising of a needle tip portion (20), a barrel portion (30) and the port portion (40). Such a needle would benefit by providing atraumatic insertion while also facilitating insertion and removal of fluids and gases and more importantly allowing passage of wire and similar structure and thus simplifying the entire process of gaining access to target location, reducing the overall time needed to perform the procedures (as fewer steps involved) and reducing chances of complications and error by reducing and simplifying the steps involved.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

### REFERENCES

Anderson, R. W. (1991, May 28). U.S. Pat. No. 5,019,039.
Johnson, G. W. (1987, Dec. 1). U.S. Pat. No. 4,710,180 A.
Pelu Tran, K. T. (2013, Dec. 17). U.S. Pat. No. 8,608,697 B2.
Peter John Crocker, M. A. (2005, Aug. 11). U.S. Pat. No. 0,177,117 A1.
Richard Kulkashi, M. P. (1992, Mar. 24). U.S. Pat. No. 5,098,388.
Teves, L. Y. (1988, Jan. 26). U.S. Pat. No. 4,721,506.
Walter A. Z0hmann. (2003, May 6). U.S. Pat. No. 6,558,353 B2.
Wiley, C. W. (2012, May 8). U.S. Pat. No. 8,172,802 B2.
Young, R. (1982, Jan. 5). U.S. Pat. No. 4,308,875.

What is claimed is:

1. A needle for use with means to transfer materials comprising (i) a tip which tapers to a point, (ii) at least one aperture partially covering the tip, in fluid communication with a conduit portion that connects to the tip portion, which is shaped to facilitate movement of wire without getting the wire caught or kinked on the aperture, (iii) at least one conduit portion (iv) at least one port and (v) where the edge of the side aperture is rounded and/or smoothened.

2. A needle according to claim 1 in which the tip has a cutting end with one or more bevels sharpened into blades

3. A needle according to claim 1 in which the tip has an atraumatic tip which is smooth and with a conical shape so that it parts tissue without cutting it.

4. A needle according to claim 1 which has an atraumatic tip which is flat so that upon penetration it pushes tissue without essentially cutting it.

5. A needle according to claim 1 in which the opening of the aperture is angled between 100 to 179 degrees measured to the longitudinal axis of the needle.

6. A needle according to claim 1 in which there is at least one conduit portion that is treated to be polished that would facilitate movement of the wire thru the needle.

7. A needle according to claim 1, further comprising a stylet or wire plug mounted with in the conduit portion to prevent particles from lodging therein.

8. A needle according to claim 1 in which, the needle tip after the last aperture towards the tip portion is solid filled in a shape to direct the wire thru aperture and not allow the wire to get stuck into the needle.

9. A needle according to claim 1, the tip portion comprising of straight or curved shape.

10. A needle for use with means to transfer materials comprising (i) a tip which tapers to a point, (ii) at least one aperture just proximal to the tip, in fluid communication with a conduit portion that connects to the tip portion, which is shaped to facilitate movement of wire without getting the wire caught or kinked on the aperture, (iii) at least one conduit portion (iv) at least one port and (v) where the edge of the side aperture is rounded and/or smoothened.

11. A needle according to claim 10 in which the tip has a cutting end with one or more bevels sharpened into blades

12. A needle according to claim 10 in which the tip has an atraumatic tip which is smooth and with a conical shape so that it parts tissue without cutting it.

13. A needle according to claim 10 which has an atraumatic tip which is flat so that upon penetration it pushes tissue without essentially cutting it.

14. A needle according to claim 10 in which the opening of the aperture is angled between 100 to 179 degrees measured to the longitudinal axis of the needle.

15. A needle according to claim 10 in which there is at least one conduit portion that is treated to be polished that would facilitate movement of the wire thru the needle.

16. A needle according to claim 10, further comprising a stylet or wire plug mounted with in the conduit portion to prevent particles from lodging therein.

17. A needle according to claim 10 in which, the needle tip after the last aperture towards the tip portion is solid filled in a shape to direct the wire thru aperture and not allow the wire to get stuck into the needle.

18. A needle according to claim 10, the tip portion comprising of straight or curved shape.

19. A method comprising:
(a) puncturing the tissue of a subject with a distal end of a hollow needle of claim 1 or 10, wherein a hollow needle having a conduit portion from the hub at the proximal end and extends through the hollow needle to the side aperture opening at the distal end, for passing wire or similar structure through the conduit portion, and wherein the aperture is angled and designed to facilitate the movement of wire in and out of needle without getting it kinked;
(b) Confirming location of needle at target location;
(c) Inserting a wire and securing the location at target location; and
(d) Removing the needle, leaving the wire at target location.

20. An tissue needle kit comprising of at least:
A tissue needle of claim 1 or 10;
A wire compatible to complement the inner diameter of the above needle.
